(19) United States
(12) Patent Application Publication
(10) Pub. No.: US 2010/0187339 A1
(43) Pub. Date: Jul. 29, 2010

(54) METHOD FOR PROCESSING MATERIAL TO PRODUCE PARTICLES OF A DESIRED SIZE

(76) Inventors: David Kannar, Victoria (AU); Barry James Kitchen, Victoria (AU)

Correspondence Address: REED SMITH LLP, P.O. BOX 488, PITTSBURGH, PA, 15230-0488, US

(21) Appl. No.: 12/161,335

(22) PCT Filed: Feb. 6, 2007
(86) PCT No.: PCT/AU2007/000116

§ 371 Date: Nov. 14, 2008

(30) Foreign Application Priority Data
Feb. 7, 2006 (AU) 2006900590

(51) Int. Cl. B02C 23/00

(52) U.S. Cl. 241/18; 241/38

(57) ABSTRACT

There is provided a method of preparing particles of a predetermined size range from material the method comprising: (A) introducing the material into a circuit comprising: (a) a mill for milling the material into particles; (b) a gas circulator for circulating a stream of gas around the circuit in which the material and the particles are entrained; and (c) a separator for separating particles of the pre-determined size range from particles greater than the pre-determined size range; (B) circulating the material to the mill; (C) milling the material to produce particles; (D) circulating the particles to the separator; (E) separating the particles into first particles having the predetermined size range and second particles having a size greater than the pre-determined size range; (F) removing the first particles from the circuit; and (G) circulating the second particles to the mill for further milling.

### FIELD OF THE INVENTION

This invention relates to methods for processing material to produce particles of a desired size and apparatus for use in the methods, including methods for processing dried material to produce coarse grain particles which produce less fine particles.

### BACKGROUND OF THE INVENTION

In this specification where a document, act or item of knowledge is referred to or discussed, this reference or discussion is not an admission that the document, act or item of knowledge or any combination thereof was at the priority date, publicly available, known to the public, part of common general knowledge; or known to be relevant to an attempt to solve any problem with which this specification is concerned.

While the following discussion relates to sugar, milk, cocoa, garlic and onions, it will be understood that the invention relates to an improved method for processing many products and that the processing may be alternatively called milling and tempering or powdering.

Products and Powders Derived from Milk.

Non fat and full fat milk powders are manufactured by spray or roller drying, followed by processes such as fluidised bed treatment to agglomerate and improve particle size distribution that ultimately improves functionality such as solubility and wettability. During these processes, particles are lecithinated to maximise these functional attributes. A number of other products are also produced as powders such as whey protein concentrates and isolates and high protein powders. Lactose is a milk sugar widely used in the food and pharmaceutical industry and many applications, particularly in pharmaceuticals, require extremely small and even particle sizes. This is usually achieved by slow and careful crystallisation of the lactose mother liquor. There is a need for a simpler and lower cost alternative to these traditional processes.

### Cocoa Processing

A key step in dry cocoa bean processing involves breaking the whole beans to release the shell (about 15% of the bean) from the centre of the bean, called the cocoa nib. This is followed by a step called winnowing where the broken pieces of nib are separated from the shell. The separation is currently carried out using the density difference and size between the nib and the shell. Sieves, shakers and airflow control are used in combination to achieve the separation so that the % shell in nib is minimized (range 1%-5% by weight). Too high shell in nib results in poor further processing and properties of the nib, which ultimately is the primary component of chocolate. There is a need for a simpler and lower cost alternative to this traditional process.

Cocoa powder is a by-product of the hydraulic pressing of cocoa liquor, the product obtained from milling and liquefying cocoa nibs. Depending upon the conditions of the press, the cocoa cake from the press, can contain from 10% to 25% cocoa fat. The cake is further milled and, in most instances, tempered to ensure that the fat is converted to a stable crystal form. Tempering is achieved by liquefying the fat (40-45° C.), then cooling to 27-29° C., followed by exiting the process for packing/bagging at 30-33° C. This ensures that the powder retains its colour during storage and subsequent use in a range of food applications. Powder tempering equipment is very expensive and takes up a lot of factory space. There is a need for a simpler and lower cost alternative to this traditional process.

### Garlic and Onions

Dry powders derived from milling dried allium species such as garlic and onion are widely used commercially as spices, flavours and therapeutic compounds. Garlic powders are thought to represent the true composition of fresh garlic more than garlic oils, extracts, pickled or pastes because the cloves are simply dried and milled.

If kept in the 50 to 70° C. temperature range with adequate airflow, the allicin yield of the dried garlic slices can be largely conserved and replicate the 90-100% allicin conserving effects of freeze drying. Temperatures above this level are thought to reduce allinase activity and therefore allicin production and so are not recommended when allicin production needs to be preserved.

Garlic and onion powders are usually produced by slicing or dicing cloves followed by static drying to a moisture content below 6%. The dried flakes are then ground to the required particle size and size distribution. The bulk density of powder products is usually between 0.690 to 0.833 grams per cubic centimetre but can be higher due to production of finer particles. Several novel drying techniques and important drying parameters have been reported in the literature (Pezzutti A, Crapiste G H. Sorptional equilibrium and drying characteristics of garlic. Journal of Food Engineering. 1991; 31:113-123.). However, little attention is given in these processes to production of larger particle sized or course grain powders. More commonly, powders produced using standard hammer milling methods contain large quantities of finer grain particles.

Many commercial manufacturers prefer to use coarse grain allium powders that do not contain a large quantity of finer particles as the larger particles flow better and are therefore easier to utilise. Tablet manufacturers, for example, prefer coarse grain powders, as they are less likely to compact during storage and transport. Compaction during transport and storage speeds oxidation and reduces the shelf life of the powder. Coarser powders also demonstrate more efficient flow characteristics through tablet-press bin-feeders and produce more consistent tablets. For these, and many other reasons, coarser grain powders are preferred.

If garlic powder is to be used in food and dietary supplements, dried garlic flake (6% or less moisture) is normally milled to reduce particle size. Some milling techniques produce excessive heat during particle reduction degrading allinase and thus allicin production. Most standard milling techniques also produce large amounts of finer grain material smaller than 80 mesh. In a typical sample for example, 100% passes through a 60 mesh screen, 75% through a 100 mesh screen, and 55% through a 115 mesh screen. This is because the dried material is brittle and breaks or shatters easily. As previously stated, fine grain powders are difficult to handle and store, so are therefore not preferred.

Earlier published approaches to deal with fines include the following:
Strittmatter (EP 613721) describes a method to dry and mill vegetable or animal materials. Wet material is discharged into a hot current of gas and hammer milled. Particles are then discharged into a turbulence chamber where material small enough is discharged and larger particles returned for further hammer milling. There is no disclosure of any method for reducing particle size wherein large particles are produced and the generation of fines is reduced.
Senseman et al (U.S. Pat. No. 2,023,247) describe a milling and drying apparatus for very high moisture content material including heavy liquid, semi-liquid or liquid/solid mixtures such as slaughterhouse blood or fruit pulp. The apparatus comprises a mill but there is no teaching of a method for reducing particle size wherein larger particles are produced and the generation of fines is reduced.
Buhler et al (EP 94810743) describes a milling and drying method to produce powdered food product. The inventors teach that drying and milling can be carried out simultaneously using wet and fibrous food products. There is no disclosure an a method for reducing particle size wherein large particles are produced and the generation of fines is reduced.
Suurnakki et al (CA 2007031) relates to a method and apparatus for producing a powder from legume feedstock. The feedstock is dehulled, crushed, preground and then ground. The powder is separated into coarse, predominantly starch based particles and fine, predominantly protein based particles. Fractionation into selected mesh sizes is not possible for all components.
Prater et al (U.S. Pat. No. 2,957,771) describes various granulation methods and equipment for garlic and onions. Prater teaches that, if a process generates large quantities of fine particles, it is feasible to aggregate these particles by moistening with water then separate the coarse grain particles. The method is applicable to recover garlic powder that has been overpulverised producing particles that are too small for commercial use. The method is therefore essentially an added recovery step to any milling or processing method producing large amounts of fines. There is no disclosure an a method for reducing particle size wherein large particles are produced and the generation of fines is reduced.
Yamamoto et al (U.S. Pat. No. 3,378,380) describes another method for producing coarse grain allium and horseradish powders. The method is divided into several stages. The first requires slicing and drying fresh bulbs to moisture content of approximately 12% using standard techniques. The dried material is then milled, screened and agglomerated at elevated temperatures using a fluidized bed of allium powder then milled. The authors claim that if this method is followed then approximately 12% of total powder produced will pass through a 100 mesh screen. This is significantly less than 40-60%, which is typically produced using standard milling equipment alone. After screening and further drying a granulated product is produced. As in the Prater patent, the agglomeration method taught by Yamamoto is essentially utilised to recover excessive fines produced during processing. There is no disclosure an a method for reducing particle size wherein large particles are produced and the generation of fines is reduced.
The agglomeration of milk powder is described by Peebles (U.S. Pat. No. 2,835,586). Like the Prater and Yamamoto patents, agglomeration is utilised to rectify the problem of over production of fines. In addition, the equipment and methods are not capable of being utilised for coarse grain allium powder production due to their high fructan content. Garlic contains over 77% carbohydrates including sugars, fructans and pectins which are sticky and viscous when moistened and cannot be handled in the manner disclosed in the Peebles patent. There is no disclosure an a method for reducing particle size wherein large particles are produced and the generation of fines is reduced.
International patent no WO 2004/066743 describes a method of processing garlic flake which contains 10-14% free moisture. At this moisture level, most milling methods and machinery would block as the high fructan and moisture combine to produce a thick viscous paste. Particles in the agglomerator have a longer transit time in the hot air stream which acts like a flash drier. Because the particles are sticky, this property is exploited and used to attract or agglomerate finer dry particles, thus a course grain or larger particle is produced. There is no disclosure an a method for reducing particle size wherein large particles are produced and the generation of fines is reduced. Milling of such low moisture material typically produces powder with a high percentage of fine particles (40 to 60%). This is because the dried material is brittle and breaks or shatters easily.
Nado Kenkyusho KK (JP 2002 709339/77) teaches the production of mulberry leaf powder by grinding and drying leaves in a rotary grinder. It is assumed the leaves have a moisture content far in excess of 8%. The process then uses a classifier followed by collection through an exhaust to product a powder, 80-90% of which is smaller than 100 mesh. There is no teaching of fractionation into selected mesh size.
Dzhambul Light Food (SU 902704) relates to equipment for drying raw materials in the production of meat and bone flour. The method provides increased productivity and improved quality by varying the deflection angle f the milling component face from 45 to 60°. The partly dried meat products typically have a moisture content of up to 50%.

Some modern pharmaceutical milling techniques can also produce coarse grain powders but, as garlic, cocoa and milk powder are low cost commodity items, these techniques are too expensive and not an option for these products. An inexpensive method capable of producing coarser grain powders would be preferred.

In addition, most milling techniques of previously dry material do not focus on maximising or conserving heat sensitive material such as allicin. An economic milling method that conserves the majority of allicin potential and produces a larger particle sized powder without generating significant numbers of fine particles is therefore desirable. Such a method would reduce the need for recovery steps inherent in the prior art and provide significant cost advantages.

### Fine Particle Production

Control of particle size is a standard part of crystalline sugar production. After granulation, dried white sugar is screened using a sloping, gyrating wire mesh screen or perforated plate. The finished refined granulated sugar is then used in food manufacturing or further milled to produce finer particle sized products such as castor or icing sugar. Particle size control of finer sugar products is often not carefully controlled which leads to a variation in the organoleptic performance of finished foods. As a result, it is often necessary for there to be additional particle size reduction of such sugar products during finished food manufacturing which leads to further costs and time to manufacture. This additional cost and time could be avoided if the sugar was more accurately produced in desired particle size ranges.

Conventional pulverizing, milling and classifying methods to produce fine particles utilize a range of technologies including a hammer mill, roller mill and fluidized bed apparatus. A fluidized bed pulverizing and classifying apparatus is capable of pulverizing a heated material by spraying compressed air from a pulverizing nozzle and causing the temperature of the fluidized bed apparatus to decrease due to the adiabatic expansion of the air. This ability makes the fluidized bed apparatus suitable for surface pulverization. The material to be pulverized enters a classifying rotor as a coarse powder and is classified as a conforming material. However, the contact between the solid materials being pulverized tends to generate a fine powder but does not typically control particle size distribution.

Japanese patent publication no. 2002-276526 discloses a one attempt to prevent over pulverization of coarse particles during fine powder production. Excessive pulverization is said to be prevented by controlling the pulverization pressure in order to reduce particle collision speeds. However, fluidization of the materials to be pulverized deteriorates when the collision speed is reduced, preventing the materials from efficiently reaching the classifying rotor and thereby still resulting in excessive pulverization due to a deterioration of the particle classification process.

U.S. Pat. No. 7,156,331 discloses a new fluidized bed pulverizing and classifying apparatus in which the behaviour of the material to be pulverized is said to be better controlled in a classifying chamber. The apparatus is reported to prevent excessive pulverization and reduce mixing of coarse and fine particles. The invention relates to a pulverizing and classifying apparatus for solids, including but not limited to, minerals, chemicals, medical substances, such as talcs, limes, ceramics, resins, cosmetics, dyes, Chinese medicines, and more particularly to a pulverizing and classifying apparatus for a toner.

There is also thus an ongoing need for a simple, inexpensive method and apparatus with a high production capacity which provides tighter control over finished particle size during fine particle production.

### SUMMARY OF THE INVENTION

It has now been found that it is possible to mill material to produce a powder which has a desired range of particle sizes, whether fine or coarse. That is, the method enables both the production of coarse grain particles having a specific particle size range and with minimal production of fine particles, as well as, the production of fine particles having a specific particle size range.

According to a first aspect of the invention, there is provided a method of preparing particles of a predetermined size range from material, the method comprising:
(A) introducing the material into a circuit comprising:
   (a) a mill for milling the material into particles;
   (b) a gas circulator for circulating a stream of gas around the circuit in which the material and the particles are entrained; and
   (c) a separator for separating particles of the predetermined size range from particles greater than the predetermined size range;
(B) circulating the material to the mill;
(C) milling the material to produce particles;
(D) circulating the particles to the separator;
(E) separating the particles into first particles having the predetermined size range and second particles having a size greater than the pre-determined size range;
(F) removing the first particles from the circuit; and
(G) circulating the second particles to the mill for further milling.

In the invention, the gas flow in the circuit is balanced with the speed of the mill so that the particles of the predetermined size are transported out of the and not further milled. Consequently the production of fines in the circuit is controlled.

Preferably, the method is continuous or semi-continuous.

Any preferred gas or mixture of gasses may be used in the method of the present invention.

Typically the gas is air. The gas could be chosen from the group comprising inert gasses such as nitrogen or argon. For example, inert gas could be used in applications where it is important to minimise oxidation of the material being milled Preferably, the gas is hepa-filtered and dehumidified. For example, if air is to be used as the gas, the air would only need to be hep-filtered and dehumidified if the material to be milled is hygroscopic or required to be of a low microbiological load.

The gas flow is carefully balanced in the machine so that particles of the desired size are adequately carried in the gas stream until reaching the separator (eg a cyclone). One example of generating adequate gas flow in the circuit is to rely on a dust collector, which has the additional benefit of reducing the speed of the mill and subsequent particle damage.

Preferably, the resulting powder collected from the separator is then directed to a sieve (typically a Sweco sieve) to recover a powder of any particle size range specification. Oversized particles can be collected in the sieve and returned to the mill for further size reduction.

While any fan or hammer mill can be used as the mill in the current invention, preferably the mill is a chopper fan wherein the chopper fan comprises fan-like plates whereby the movement of the plates assists in circulating the gas stream. The chopper fan acts as a hammer mill but is also used to reduce damage to the incoming material.

Preferably, the circuit is a closed ducting circuit.

Preferably, the gas is dry air. But inert gases can be used if the material is particularly sensitive to oxidation.

Depending on the material to be processed in the method of the invention, the gas is conditioned to match the properties of the material. The gas may need be heated or cooled to an appropriate temperature. If the material is hygroscopic, then the gas may be heated to remove water content. For heat sensitive materials, the gas temperature is chosen so that denaturation or damage is minimized. For materials having a low melting point, the gas may be cooled to ensure the materials are kept amorphous and/or crystalline.

The method of the invention can either be run on a batch or continuous basis depending upon the quantity of material to be processed.

The desired range of particle sizes will depend on the material and finished particle size which can be largely controlled by sieve mesh size. For example, a fine particle sized sugar product between 63-125 μm can be produced using a 100 mesh screen. As another example, a coarse grain garlic powder between 60-125 μm can be produced using a 20 mesh screen.

The method of the invention can be used in relation to any material including synthetic compounds, drugs and foods, especially those whose active compounds are heat sensitive or produced by enzyme hydrolysis. Examples of appropriate foods include vegetables, fruit, sugar, cocoa. In a particularly preferred embodiment the material milled is food that can be freeze dried.

Preferably, where the material contains one or more active compounds which are heat sensitive (the activity of the active compound is reduced by heating), the activity of the active compound is substantially retained relative to the activity of the active compound in the original material. The term “substantially retained” means that the activity of the active compound in the final particles is at a level of at least 50% compared to the activity of active compound in the original material. Preferably, the activity is at least 60%, more preferably 70%, even more preferably 80%, most preferably 90% or 95%.

Examples of such active compounds include the group consisting of flavours, pharmaceutical compounds, pharmaceutical excipients, plant compounds, enzymes, polysaccharides, gums, mucilages, starches and proteins. Examples of material containing active compounds which are heat sensitive include the group consisting of garlic, onion, horseradish, cocoa, fruit and grape extracts.

The method of the invention could be used to process (milling and tempering) cocoa cake to produce cocoa powder. Once milled to particles of an appropriate size, the cocoa particles would pass through additional ducting having different temperatures of air intake so that the cocoa powder can pass through a variety of temperatures (for example, 40-45° C. then cooling to 27-29° C., followed by 30-33° C.).

For milk powders, lecithin or another appropriate emulsifier can be sprayed into the ducting after the particles have been milled to an appropriate size using the method of the invention. By controlling the humidity in the in-feed and injecting a fine spay of a 1-5% lecithin suspension at the same time, milk powder particles can be coated prior to cooling and separation in the sieving portion of the equipment. Products such as whey protein concentrates and isolates, as well as high protein powders, would also have their functionality improved by milling and sieving in the equipment.

The method according to the invention could also be used to mill coarse lactose crystals and separate them into a consistently small particle size suitable for a wide range of applications in both the food and pharmaceutical industries. The ability to remove the water content from the gas is important as pharmaceutical lactose is hygroscopic.

Using the invention, it is now also possible to mill dried material to produce a powder which has larger particles and thus improved handling properties, whilst at the same time the activity of heat-sensitive active compounds is substantially retained. Particles prepared using this method can be more easily incorporated into other products using standard manufacturing equipment without the difficulties associated with handling fine particles. In this embodiment of the invention, the dried material typically has a free moisture content level of 6% or less. Garlic flake or vegetables freeze dried typically contain 4-5% free moisture to preserve the material. Milk powders and cocoa powders usually contain between 1-3% free moisture.

According to a one preferred embodiment of the invention, there is provided a method of preparing coarse particles of a predetermined size range from dried material having a moisture content of 6% or less, said method comprising:
(A) introducing the dried material into a circuit comprising:
   (a) a mill for milling the dried material into coarse particles;
   (b) a gas circulator for circulating a stream of gas around the circuit in which the dried material and the coarse particles are entrained; and
   (c) a separator for separating coarse particles of the pre-determined size range from particles greater than the pre-determined size range;
(B) circulating the material to the mill;
(C) milling the material to produce particles;
(D) circulating the particles to the separator;
(E) separating the particles into first particles having the predetermined size range and second particles having a size greater than the predetermined size range;
(F) removing the first particles from the circuit; and
(G) circulating the second particles to the mill for further milling;
   wherein the gas is hepa-filtered and dehumidified; and
   wherein the gas flow in the circuit is balanced with the speed of the mill so that the coarse particles of the predetermined size range are transported out of the mill therefore reducing production of fine particles.

The advantage of this preferred embodiment of the method of the invention is that coarse grain particles are prepared with a minimum of fines. The coarse grain particles will need to conform to standards for the particular dried material being processed. For example, coarse grain garlic particles should to conform American Dehydrated Onion and Garlic Association which specifies a range of mesh sizes from 40 to 100 (400 to 160 microns). Coarse grain particles prepared by the method of the present invention are also provided.

For this preferred embodiment, preferably, the particles separated from the circuit are of a size distribution such that less than 40%, preferably 30% or 20%, most preferably 10% or 5% of the particles will pass through a 100 mesh sieve. That is, there is minimum production of fine particles.

This preferred embodiment of the method of the invention could be used to carry out the cracking and winnowing process for cocoa beans to produce cocoa nib having a low shell in nib content. The balance of the gas flow with the speed of the mill (typically called a grinder/cracker in this context) in addition to the use of the most appropriate sieve size and vibration is expected to provide low shell in nib content.

The finished powder is valuable for production of tablets, capsules, dietary supplements and foods. The method has the advantage of easier and more economic production of powder over present grinding processes that produce a large proportion of fine particles that must be sieved out and agglomerated or used in lower value applications. Preferably the particle size of the finished powder used to make pharmaceutical dose forms such as tablets, is not less than 100 mesh and the moisture content is about 5% dry weight. The preferred results will however be dependent upon requirements of the end user.

The powder may be presented in tablet form. It will be readily understood by those skilled in the art that the powder can be formulated a number of different ways. It will be understood that a variety of different binders, fillers and a number of other excipients can be used. An enteric coating may also be applied to reduce acidic degradation during intestinal transit. The enteric coating is usually applied using standard methods and may include cellulose, methylcellulose or a derivative of either of these or another similar substance designed to delay the release of the active ingredients. One method that can be used is that cited in international patent publication WO 01/76392. It is also possible to place the powder in other delayed release delivery systems for delivering the powder to the small intestine. Typically, the delivery systems will however comply with standards specified for delayed release dose forms in the USP 2000.

According to a second aspect of the invention, there is provided an apparatus for preparing particles having a predetermined size ranger from a material, said apparatus comprising a circuit comprising:
(a) a mill for milling the material into particles;
(b) a gas circulator for circulating a stream of gas around the circuit in which the material and the particles are entrained; and
(c) a separator for separating particles of the predetermined size range from particles greater than the predetermined size range;
wherein the gas flow in the circuit is balanced with the speed of the mill so that the particles of the predetermined size range are transported out of the mill thereby reducing the production of particles outside the desired particle size range.

According to a third aspect of the invention, there is provided particles having a predetermined size range produced by introducing material into a circuit comprising:
(a) a mill for milling the material into particles;
(b) a gas circulator for circulating a stream of gas around the circuit in which the material and the particles are entrained; and
(c) a separator for separating particles of the predetermined size range from particles greater than the predetermined size range;
   wherein the gas flow in the circuit is balanced with the speed of the mill so that the particles of the predetermined particle size range are transported out of the mill thereby reducing the production of particles outside the predetermined particle size range.

### BRIEF DESCRIPTION OF THE FIGURES

FIG. 1 is an illustration of a milling machine used in one embodiment of the invention.

FIG. 2 shows the average particle size by micrometer analysis for the commercial castor sugar compared to the castor sugar milled according to the invention in Example 3.

FIG. 3 shows the average particle size by micrometer analysis for the commercial refined white sugar compared to the white sugar milled according to the invention in Example 3.

### DETAILED DESCRIPTION OF A PREFERRED EMBODIMENT

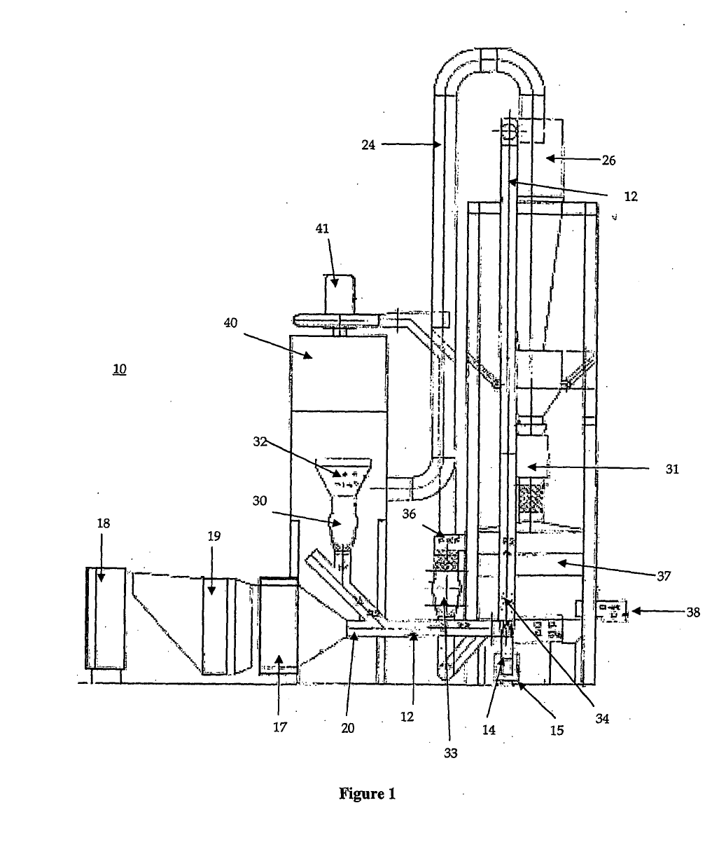

FIG. 1 depicts views of a circuit 10 according to a preferred embodiment of the present invention. The circuit comprises circuit ducts 12, a feeder and rotating airlock 30, a chopper fan 14, and an extraction duct 24. The chopper fan 14 is driven by a motor 15. A dehumidifier 18, hepa filter 19, and heat exchanger 17 heat the air to an operating temperature. The heated air circulates through the ducts 12 as a heated gas stream 20 in direction 22 towards the cyclone 26. The circulation may be effected by any suitable means, such as a vacuum or the like, but is preferably effected by an extraction fan 41 attached to a dust collector 40.

In operation, dried material 32 is fed into the circuit ducts 12 via a feeder and rotating air lock 30. The material 32 is transported to the chopper fan 14 where it is milled into smaller particles 34 in the heated gas stream 20. The particles 34 are then transported by the heated gas stream 20 to the cyclone 26, then directed via a feeder and rotating valve 31 to a sieve 37 (typically a Sweco sieve) where the milled particles of a pre-determined size 38 are separated and collected. Particles greater than the pre-determined size 36 are redirected back to the chopper fan 14 via a rotating valve 33 and ducts 12.

In addition to reducing particle size, the chopper fan 14 acts as a hammer mill which is also capable of promoting adequate movement of the circulating air load.

### EXAMPLES

In order that the nature of the present invention may be more clearly understood, preferred forms thereof will now be described with reference to the following non-limiting examples.

### Example 1

The ability of the method according to the invention to produce a course grain powder was investigated in this example.

### Material

Dried Garlic Flake: Garlic flake 4-6% moisture

### Chopper Fan: 17 Hz

Dust Collector Airflow 22 m/sec

### Test Method:

A filtered dry air load was established in the circuit using a dehumidifier, hepa-filter, and heater attached to the air intake and starting the chopper and suction fans. When the inlet air temperature had reached approximately 50° C. 1 kg of dried garlic flake was then fed into the feed and rotating valve located immediately after the heater at the rate of approximately 0.5 kg per minute.

### Results:

The finished garlic powder was split using a Sweco sieve into product passing through 20 mesh screen and product passing through a 100 mesh screen. Product passing through the 20 mesh screen but not the 100 mesh screen was weighed as finished product. Product passing through the 100 mesh screen was considered fines.

TABLE 1

| Sieve size | % product passing through |
|---|---|
| 100 mesh | 15% fines |
| 20 mesh | 100% (85% is finished product) |

### Conclusion:

This data provides proof that the method according to the this invention is capable of producing a course grain powder without the significant loss of fines produced with standard milling equipment (40 to 60%).

### Example 2

In order to determine the optimal operating conditions of the apparatus to produce minimal fines and balance airflow between the returning duct and ascending duct, speed of the chopper fan and dust collector were varied as follows.

### Material

Dried Garlic Flake: 100 g garlic flake samples of 4-6% moisture were fed into the mill. Finished garlic powder was split using a Sweco sieve into product passing through 20 mesh screen and product passing through a 100 mesh screen. Product passing through the 20 mesh screen but not the 100 mesh screen was weighed as finished product. Product passing through the 100 mesh screen was considered fines.

### Test Method:

A filtered dry air load was established in the circuit using a dehumidifier, hepa-filter, and heater attached to the air intake and starting the chopper and suction fans. When the inlet air temperature had reached approximately 50° C., 100 gm samples of dried garlic flake was fed into the feed and rotating valve located immediately after the heater.

### Dust Collector Speed: 50 Hz

### Results:

TABLE 2

| Chopper fan (Hz) | Chopper fan (rpm) | Finished Product | Fines |
|---|---|---|---|
| 17 | 960 | 70% | 30% |
| 20 | 1124 | 76% | 23% |
| 25 | 1400 | 83% | 17% |
| 30 | 1700 | 84% | 16% |
| 32 | 1800 | 85% | 15% |

### Conclusion:

This experiment demonstrated that the most efficient settings to improve efficiency of the mill and reduce fines was to run the chopper fan at 1800 rpm and dust collector at 50 Hz.

At these settings, air volume measured was balanced or the same at the returning duct and ascending duct. Without being bound by theory, it is believed that balancing airflow reduces production of fines in the milling machine by reducing cavitation around the chopper fan. Airflow measured at the ascending duct was approximately 760 m/s which appeared to improve efficiency of the cyclone.

### Example 3

The following example demonstrates use of the method of the invention in industrial sugar manufacturing to produce a fine grade sugar product with increased control of the particle size range.

First, commercial castor sugar was analysed for its particle size distribution. Then a 2 kg sample of the castor sugar was processed according to the method of the invention using the mill shown in FIG. 1 using an airflow range from 20 to 30 m/s and temperature range from 5 to 50° C. It is noted that the mill can be run up to about 110° C., the operating temperature depending upon the material being milled. For example, when milling sugar using the method of the present invention, ambient temperatures would be used and dehumidified, hepa filtered air used as the gas.

A 100 g sample of each material was then analysed with a Fritsch Vibratory Sieve Shaker Analysette 3 SPARTAN Pulverisette at amplitude 3 mm for 20 minutes. The results are shown in tables 3 and 4.

TABLE 3

| | | Weight share of fractions, wt % | | | | | | | |
|---|---|---|---|---|---|---|---|---|---|
| | | Fractions, μm | | | | | | | |
| | | | | | | | | | Total |
| | | | | | | | | | weight of |
| Test | Material | 425 | 250-425 | 125-250 | 90-125 | 63-90 | 38-63 | <38 | fractions, % |
| 1 | Commercial | 30.51 | 50.84 | 15.59 | 0.8 | 0.7 | 0 | 0 | 98.44 |
| | Castor Sugar | | | | | | | | |
| 2 | 2 kg Castor | 0 | 0 | 0 | 33.59 | 23.99 | 37.53 | 3.40 | 98.51 |
| | Sugar, run for 5 min, | | | | | | | | |
| | 80% | | | | | | | | |
| | produced in 3 min, | | | | | | | | |
| | using the | | | | | | | | |
| | present invention | | | | | | | | |

TABLE 4

| | | *Average particle size by micrometer, μm | | | | | | | |
|---|---|---|---|---|---|---|---|---|---|
| | | Fractions, μm | | | | | | | |
| | | | | | | | | | Sample |
| | | | | | | | | | before |
| Test | Material | >425 | 250-425 | 125-250 | 90-125 | 63-90 | 38-63 | <38 | sieving |
| 1 | Unprocessed | 465 | 312 | 196 | 120 | 82 | — | — | 432 |
| | Castor Sugar | | | | | | | | |
| 2 | Castor Sugar, run | — | — | — | 110 | 72 | 31 | 23 | 74 |
| | for 5 min, using | | | | | | | | |
| | the present | | | | | | | | |
| | invention | | | | | | | | |

*Average particle size by micrometer analysis has been carried out at 0.5 sugar/paraffin ratio (weight).

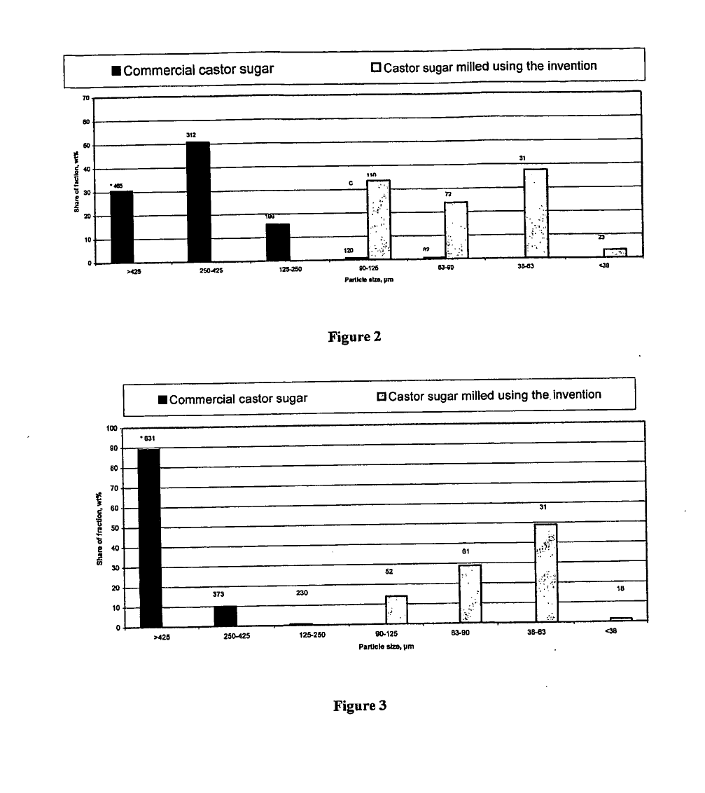

FIG. 2 shows the average particle size by micrometer analysis for the commercial castor sugar compared to the castor sugar milled according to the invention. The average particle size for each fraction is shown at the top of each column. The average particle size for the unsieved samples was 432 μm for the unprocessed castor sugar and 74 μm for the milled castor sugar.

Second, commercial white refined sugar was analysed for its particle size distribution. Then a 10 kg sample of the refined white sugar was processed according to the method of the invention using the mill shown in FIG. 1 using an airflow range from 20 to 30 m/s at ambient temperature of 23° C. (intake temperature 31° C.; outlet temperature 36° C.).

A 100 g sample of each material was then analysed with a Fritsch Vibratory Sieve Shaker Analysette 3 SPARTAN Pulverisette at amplitude 3 mm for 20 minutes. The results are shown in tables 5 and 6.

TABLE 5

| | | Weight share of fractions, wt % | | | | | | | |
|---|---|---|---|---|---|---|---|---|---|
| | | Fractions, μm | | | | | | | |
| | | | | | | | | | Total |
| | | | | | | | | | weight of |
| Test | Material | >425 | 250-425 | 125-250 | 90-125 | 63-90 | 38-63 | <38 | fractions, % |
| 1 | Unprocessed | 89.21 | 9.97 | 0.65 | 0 | 0 | 0 | 0 | 99.83 |
| | Refined White | | | | | | | | |
| | Sugar | | | | | | | | |
| 2 | 10 kg Refined | 0 | 0 | 0 | 13.95 | 28.84 | 48.90 | 1.39 | 93.08 |
| | White Sugar | | | | | | | | |

TABLE 6

| | | *Average particle size by micrometer, μm | | | | | | | |
|---|---|---|---|---|---|---|---|---|---|
| | | Fractions, μm | | | | | | | |
| | | | | | | | | | Sample |
| | | | | | | | | | before |
| Test | Material | >425 | 250-425 | 125-250 | 90-125 | 63-90 | 38-63 | <38 | sieving |
| 1 | Unprocessed | 631 | 373 | 230 | — | — | — | — | 619 |
| | Refined White | | | | | | | | |
| | Sugar | | | | | | | | |
| 2 | 10 kg Refined | — | — | — | 52 | 61 | 31 | 18 | 43 |
| | White Sugar | | | | | | | | |

*Average particle size by micrometer analysis has been carried out at 0.5 sugar/paraffin ratio (weight).

FIG. 3 shows the average particle size by micrometer analysis for the commercial refined white sugar compared to the white sugar milled according to the invention. The average particle size for each fraction is shown at the top of each column. The average particle size for the unsieved samples was 619 μm for the unprocessed white sugar and 43 μm for the milled white sugar.

### CONCLUSION

The above results demonstrate that a smaller particle size within a narrower range compared to commercial castor sugar, can be produced using the method and machinery described in the current invention. For example in excess of 95% of castor sugar milled (Table 3) was between 38-125 μm which was smaller than commercial castor sugar of which greater than 30% had a particle size in excess of 425 μm and a greater distribution of particle size ranging from <425 to 63 μm.

The word ‘comprising’ and forms of the word ‘comprising’ as used in this description and in the claims does not limit the invention claimed to exclude any variants or additions.

Modifications and improvements to the invention will be readily apparent to those skilled in the art. Such modifications and improvements are intended to be within the scope of this invention.

1. A method of preparing particles of a predetermined size range the method comprising:
(A) introducing the material into a circuit comprising:
(a) a mill for milling the material into particles;
(b) a gas circulator for circulating a stream of gas around the circuit in which the material and the particles are entrained; and
(c) a separator for separating particles of the pre-determined size range from particles greater than the pre-determined size range;
(B) circulating the material to the mill;
(C) milling the material to produce particles;
(D) circulating the particles to the separator;
(E) separating the particles into first particles having the predetermined size range and second particles having a size greater than the pre-determined size range;
(F) removing the first particles from the circuit; and
(G) circulating the second particles to the mill for further milling.

2. The method according to claim 1 when operated continuously or semi-continuously.

3. The method according to claim 1 wherein step (E) includes sieving the particles in a sieve to recover the first particles.

4. The method according to claim 1 wherein step (E) includes separating the particles in a cyclone.

5. The method according to claim 1 wherein step (C) includes chopping the material in a chopper fan.

6. The method according to claim 1 wherein the gas is hepa-filtered and dehumidified.

7. The method according to claim 1 wherein the gas is dry air.

8. The method according to claim 1 wherein the gas is conditioned to match the properties of the material.

9. The method according to claim 1 wherein the material is selected from the group consisting of garlic, onion, horseradish, cocoa, fruit and grape extracts, sugar, milk powders or extracts, lactose and mixtures thereof.

10. A method of preparing coarse particles of a predetermined size range from dried material having a moisture content of 6% or less, said method:
(A) introducing the dried material into a circuit comprising:
(a) a mill for milling the dried material into coarse particles;
(b) a gas circulator for circulating a stream of gas around the circuit in which the dried material and the coarse particles are entrained; and
(c) a separator for separating coarse particles of the pre-determined size range from particles greater than the pre-determined size range;
(B) circulating the material to the mill;
(C) milling the material to produce particles;
(D) circulating the particles to the separator;
(E) separating the particles into first particles having the predetermined size range and second particles having a size greater than the predetermined size range;
(F) removing the first particles from the circuit; and
(G) circulating the second particles to the mill for further milling;
wherein the gas is hepa-filtered and dehumidified; and
wherein the gas flow in the circuit is balanced with the speed of the mill so that the coarse particles of the predetermined size range are transported out of the mill therefore reducing production of fine particles.

11. The method according to claim 10 wherein the dried material has a free moisture content of 6% or less.

12. The method according to claim 10 wherein the particles having the predetermined size range are of a size distribution such that less than 40% of the particles will pass through a 100 mesh sieve.

13. The method according to claim 12 wherein less than 20% of the particles will pass through a 100 mesh sieve.

14. The method according to claim 13 wherein less than 5% of the particles will pass through a 100 mesh sieve.

15. The method according to claim 10 wherein the dried material is selected from the group consisting of garlic, onion, horseradish, cocoa, fruit and grape extracts, sugar, milk powders or extracts, lactose, cocoa bean and mixtures thereof.

16. An apparatus for preparing particles having a predetermined size range from a material, said apparatus comprising a circuit comprising:
(a) a mill for milling the material into particles;
(b) a gas circulator for circulating a stream of gas around the circuit in which the material and the particles are entrained; and
(c) a separator for separating particles of the predetermined size range from particles greater than the predetermined size range;
wherein the gas flow in the circuit is balanced with the speed of the mill so that the particles of the predetermined size range are transported out of the mill thereby reducing the production of particles outside the desired particle size range.

17. The mill according to claim 16 further comprising a sieve to recover particles of the predetermined size range.

18. Particles having a predetermined size range produced by introducing material into a circuit comprising:
(a) a mill for milling the material into particles;
(b) a gas circulator for circulating a stream of gas around the circuit in which the material and the particles are entrained; and
(c) a separator for separating particles of the predetermined size range from particles greater than the predetermined size range;
wherein the gas flow in the circuit is balanced with the speed of the mill so that the particles of the predetermined particle size range are transported out of the mill thereby reducing the production of particles outside the predetermined particle size range.
